SEQUENCE LISTING (6 sequences)

SEQ ID NO: 1 - DNA, 41 bp, Artificial Sequence
  Synthetic Primer
ttggattgcagtagcgggatgtgcatgcgtgcagattcgtc

SEQ ID NO: 2 - DNA, 38 bp, Artificial Sequence
  Synthetic Primer
cgcaaagtcgcgcgatggtaacggatcacaccatggca

SEQ ID NO: 3 - DNA, 17 bp, Artificial Sequence
  Synthetic Primer
ccgcatacgtgttggca

SEQ ID NO: 4 - DNA, 18 bp, Artificial Sequence
  Synthetic Primer
tgcgttttgatggtgcct

SEQ ID NO: 5 - DNA, 24 bp, Artificial Sequence
  Synthetic Primer
acggaagaatcgagggttttgtac

SEQ ID NO: 6 - DNA, 18 bp, Artificial Sequence
  Synthetic Primer
gtcaccgtccggaccgtg

(19) United States
(12) Patent Application Publication
(10) Pub. No.: US 2018/0080067 A1
(43) Pub. Date: Mar. 22, 2018

(54) RAPID PERTUSSIS DIAGNOSIS ON A POINT-OF-CARE HYBRID MICROFLUIDIC BIOCHIP

(71) Applicants: XiuJun Li, El Paso, TX (US); Maowei Dou, El Paso, TX (US); Delfina C. Dominguez, El Paso, TX (US)

(72) Inventors: XiuJun Li, El Paso, TX (US); Maowei Dou, El Paso, TX (US); Delfina C. Dominguez, El Paso, TX (US)

(73) Assignee: THE BOARD OF REGENTS OF THE UNIVERSITY OF TEXAS SYSTEM, Austin, TX (US)

(21) Appl. No.: 15/701,886

(22) Filed: Sep. 12, 2017

Related U.S. Application Data
(60) Provisional application No. 62/394,350, filed on Sep. 14, 2016.

(51) Int. Cl. C12Q 1/68; B01L 7/00; B01L 3/00

(52) U.S. Cl. CPC C12Q 1/689; B01L 7/00; B01L 3/5027; B01L 2200/10; B01L 2200/16; B01L 2300/1827; B01L 2300/0816; B01L 2300/126

(57) ABSTRACT

Certain embodiments are directed to a point-of-care (POC) microfluidic biochip for rapid, highly sensitive and specific pertussis diagnosis. The POC biochip can be used in various venues such as physician's office, schools, hospitals, and low-resource settings so that rapid prevention and treatment of pertussis can be achieved. The POC biochip based pertussis diagnosis is low-cost and does not rely on any specialized instrument, but offers comparable sensitivity to real-time PCR.

### PRIORITY PARAGRAPH

This application claims priority to U.S. Provisional Application 62/394,350 filed Sep. 14, 2016, which is incorporated herein by reference in its entirety.

### REFERENCE TO SEQUENCE LISTING

A sequence listing required by 37 CFR 1.821-1.825 is being submitted electronically with this application. The sequence listing is incorporated herein by reference.

### STATEMENT REGARDING FEDERALLY FUNDED RESEARCH

This invention was made with government support under R21AI107415 awarded by the NIH/NIAID. The government has certain rights in the invention.

### BACKGROUND

Pertussis, also known as whooping cough, is a contagious disease that is caused by the bacterium Bordetella pertussis (B. pertussis). Despite the high vaccination coverage in many countries for more than 50 years, the vaccine-preventable pertussis remains endemic worldwide (Kilgore et al., Clinical Microbiology Reviews 2016, 29, 449-486; Organization, W. H. World Health Organization, Geneva, Switzerland. URL=apps.whaint/iris/bitstream/10665/70149/1/WHO_IVB_2009_eng.pdf 2009). According to a report from the World Health Organization (WHO), there were about 16 million pertussis cases worldwide in 2008 with 95% of these pertussis causes occurred in developing countries, and there were about 195,000 deaths from the disease (Organization, W. H. World Health Organization, Geneva, Switzerland. URL=who.int/immunization/topics/pertussis/en/index.html 2011). Pertussis is also a common disease in the United States with frequent outbreaks. For example, in the most recent peak year of 2012, 48,277 cases of pertussis were reported, which was the highest level since 1959 (Faulkner et al., VPD Survelliance Manual 2011, 1-12).

Pertussis is commonly under diagnosed because most cases present as mild or subclinical infectious (Singh and Lingappan, CHEST Journal 2006, 130, 1547-1553; Wendelboe and Van Rie, Expert review of molecular diagnostics 2006, 6, 857-864). Pertussis presents with a runny nose, mild cough and low-grade fever. Those syndromes caused by pertussis and the other respiratory infections such as Respiratory Syncytial Virus (RSV), rhinovirus, Mycoplasma pneumoniae and Chlamydophila pneumoniae are often indistinguishable especially during the winter season (Pierce et al., Journal of clinical microbiology 2011, JCM-05996). And only when the cough becomes persistent or prominent do clinicians tend to suspect pertussis. Furthermore, patients infected with pertussis sometimes may not present with any significant syndromes (Munoz, Seminars in pediatric infectious diseases 2006, 17, 14-19). Given that pertussis is highly infectious during the acute phase (10 days) of infection, it is important to diagnose and confirm suspected cases as soon as possible and to limit contact with high-risk populations such as infants and the elderly, who are more vulnerable to serious infections and complications.

Bacterial culture and molecular testing such as real-time polymer chain reaction (qPCR) are the current diagnosis methods of pertussis. However, culture is insensitive and slow (>7 days) (Fry et al., Journal of medical microbiology 2004, 53, 519-525). qPCR has high sensitivity but requires costly instrumentation (e.g., qPCR ˜$60000). Serological testing is also available for diagnosis but its sensitivity and specificity is low (Orenstein, Clinical infectious diseases 1999, 28, S147-S150). In addition, multiple variables including test systems, delays in specimen collection and transportation to the laboratory, costly instrumentation and lack of personnel expertise may affect laboratory diagnosis of pertussis (Cherry et al., The Pediatric infectious disease journal 2005, 24, S25-S34).

The microfluidic lab-on-a-chip technique developed in 1990s has recently offered a unique platform for various biomedical applications, allowing for low reagent consumption, fast and sensitive analysis, high portability, and integrated processing and analysis of complex biological fluids with high efficiency (Dou et al., Talanta 2015, 145, 43-54; Sanjay et al., Analyst 2015, 140, 7062-7081; Liu et al., Lab on a Chip 2011, 11, 1041-1048; Shen et al., Biomicrofluidics 2014, 8, 014109; Li et al., Bioanalysis 2012, 4, 1509-1525; Li and Zhou, Microfluidic devices for biomedical applications; Elsevier, 2013). Recently, microfluidic chips integrated with loop-mediated isothermal amplification (LAMP) have been developed for rapid detection of various pathogens such as S. aureus, E. coli, and M. tuberculosis (Fang et al., Lab on a Chip 2012, 12, 1495-1499; Safavieh et al., Analyst 2014, 139, 482-487; Safavieh et al., Biosensors and Bioelectronics 2012, 31, 523-528; Wang et al., Lab on a Chip 2011, 11, 1521-1531; Fang et al., Analytical Chemistry 2010, 82, 3002-3006; Dou et al., Analytical chemistry 2014, 86, 7978-7986). Although those microfluidics based LAMP approaches demonstrated various benefits including low cost, low reagent consumption, portability and rapidness, the potential application for POC detection of pathogens are still hard to be achieved. For one thing, instead of using real clinical samples, the feasibilities of those on-chip LAMP approaches were proved using extracted DNA. However, the clinical sample preparations usually rely on centrifuges and their procedures are complicated and time-consuming, which may not compatible with those approaches. For another, external supplies (e.g., centrifuges or water baths), are still needed. But those supplies are usually not available for in-filed diagnosis or in low-resource settings.

A low-cost point-of-care (POC) device for rapid, highly-sensitive and specific diagnosis of pertussis is urgently needed.

### SUMMARY

The inventors have developed a POC microfluidic biochip for rapid, highly sensitive and specific pertussis diagnosis. The POC biochip can be used in various venues such as physician's office, schools, hospitals, and low-resource settings so that rapid prevention and treatment of pertussis can be achieved. The POC biochip based pertussis diagnosis is low-cost, rapid, highly sensitive and specific. To address the problems outlined above, a biochip for POC analysis and related methods has been developed for detecting B. pertussis. In certain aspects the system does not rely on external power supplies. Certain embodiments are directed to a device having at least one microwell or viewing window that is configured so that a signal generated is detectable by the human eye, i.e., a signal is detectable upon visual inspection. In further aspects a mobile computing device can be used as a detector or image capture device. In certain aspects a smartphone or tablet camera can be used as a detector or to capture an image. In a further embodiment the signal can be quantitated using a portable, battery-powered spectrophotometric system. In certain aspects the system can have a detector, configured to detect light.

The device can further comprise one or more amplification zones. In certain aspect at least one amplification zone comprises B. pertussis specific amplification primers. In a further aspect the amplification primers can be selected from primers having or consisting essentially of a nucleotide sequence of SEQ ID NO:1, SEQ ID NO:2, SEQ ID NO:3, SEQ ID NO:4, SEQ ID NO:5, and/or SEQ ID NO:6.

In other aspects the system can further comprise a microfluidic device incubator or heating element configured to develop assay reagents that are applied to or included in the microfluidic device. In certain aspects the assay reagents are nucleic acid amplification reagents. In a further aspect the assay reagents are LAMP reagents, wherein a developed LAMP reaction produces a detectable signal upon the presence of a target nucleic acid. In certain aspect the amplification reagents can be B. pertussis specific amplification reagents, including B. pertussis specific amplification primers. In a further aspect the amplification primers can be selected from primers having a nucleotide sequence of TTGGATTGCAGTAGCGGGATGTGCATGCGTGCAGATTCGTC (SEQ ID NO:1); CGCAAAGTCGCGCGATGGTAACGGATCACACCATGGCA (SEQ ID NO:2); CCGCATACGTGTTGGCA (SEQ ID NO:3); TGCGTTTTGATGGTGCCT (SEQ ID NO:4); ACGGAAGAATCGAGGGTTTTGTAC (SEQ ID NO:5); and/or GTCACCGTCCGGACCGTG (SEQ ID NO:6). In certain aspects the microfluidic device incubator is a heater.

Other embodiments are directed to methods of detecting B. pertussis in a sample comprising: introducing a sample suspected of having B. pertussis bacteria into a microfluidic device configured to specifically amplify B. pertussis DNA; incubating the microfluidic device at a temperature for LAMP amplification of B. pertussis DNA; and detecting the presence or absence of B. pertussis DNA based on DNA amplification. In certain aspects the LAMP amplification temperature is 50° C., 55° C. to 60° C., 65° C., 70° C. (including all values and ranges there between), preferably about 65° C. The microfluidic device can be configured with a positive control and a negative control for B. pertussis DNA amplification. The method can further comprise exposing the sample to a bacterial lysis solution, preferably prior to LAMP amplification.

The phrase “specifically binds” or “specifically immunoreactive” to a target refers to a binding reaction that is determinative of the presence of the molecule, microbe, or other targets in the presence of a heterogeneous population of other biologics. Thus, under designated conditions, a specified molecule binds preferentially to a particular target and does not bind in a significant amount to other biologics present in the sample.

As used herein, the term “test sample” generally refers to a material suspected of containing one or more targets. The test sample may be used directly as obtained from the source or following a pretreatment to modify the character of the sample. The test sample may be derived from any biological source, such as a physiological fluid, including, blood, interstitial fluid, saliva, ocular lens fluid, cerebral spinal fluid, sweat, urine, milk, ascites fluid, mucous, lung secretions, synovial fluid, peritoneal fluid, vaginal fluid, amniotic fluid or the like. The test sample may be pretreated prior to use, such as preparing plasma from blood, diluting viscous fluids, and the like. Methods of treatment may involve filtration, precipitation, dilution, distillation, mixing, concentration, inactivation of interfering components, and the addition of reagents. Besides physiological fluids, other liquid samples may be used such as water, food products, and the like for the performance of environmental or food production assays. In addition, a solid material suspected of containing the target may be used as the test sample. In some instances it may be beneficial to modify a solid test sample to form a liquid medium or to release a target.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1a-c. The PDMS/paper hybrid microfluidic biochip. (a) A photograph; (b) The biochip layout; (c) A cross-section view of the LAMP zone illustrating the detection principle.

FIGS. 2a-d. Design of the portable battery-powered heater. (a) 3D schematic of the holder for the heater; (b) A photograph of the heater; (c) Schematic of the PID-based temperature controller; (d) DC variable voltage heating rate curve.

FIGS. 3a-b. Off-chip LAMP products of PC and NCs after the LAMP reaction under daylight (a) and a portable UV light (b). The PC tube showed green color and bright green fluorescence. Neither the NC tube showed notable color change or fluorescence. PC: LAMP product of B. pertussis; NC 1: no template DNA; NC 2: no LAMP primers.

FIGS. 4a-e. On-chip LAMP reaction and detection of B. pertussis using purified DNA by a portable UV light pen (a) and fluorescence microscopy (b). Strong fluorescence was observed in B. pertussis and PC LAMP zones, but not in NC zones. (c) Gray value of the LAMP products measured by ImageJ; (d) Fluorescent intensity of the LAMP products measured by fluorescence microscope. (e) Gel electrophoresis of on-chip LAMP products. Lanes 1-3: 100 bp ladder, B. pertussis LAMP products, NC. Ladder-pattern DNA bands were observed in B. pertussis products, whereas no DNA bands were observed in NC. The purified DNA template used was 5×10⁶ copies per LAMP zone.

FIGS. 5a-c. Specificity study among B. pertussis, B. parapertussis and B. holmesii. Fluorescence images of on-chip LAMP products to test specificity between B. pertussis and B. parapertussis (a) and specificity between B. pertussis and B. holmesii (b). Only the LAMP zones with B. pertussis template DNA showed bright fluorescent signal, whereas LAMP zones loaded with B. parapertussis and B. holmesii template DNA showed similar signal to NC. (c) Gel electrophoresis of the on-chip LAMP products for confirmatory analysis. Lane 1: 100 bp marker; Lanes 2-3: products of B. pertussis and B. parapertussis LAMP zones from (a); Lanes 4-5: LAMP products of B. pertussis and B. holmesii LAMP zones from (b).

FIGS. 6a-c. LOD investigation. (a) Fluorescence images of LAMP products using a series of 10 fold diluted B. pertussis DNA template solutions ranging from 50, 5.0 and <1 DNA copies per LAMP zone, as well as the NC. The on-chip LAMP products still exhibited strong fluorescence even the initial DNA templates were as low as 5 copies per LAMP zone. (b) Gray values of the image of (a) for LAMP products from 50, 5.0 and <1 DNA copies of DNA template per LAMP zone, as well as the NC. The dotted line is the calculated gray value (35.5) of the cutoff line for B. pertussis detection based on 3 times SD of negative controls. (c) Gel electrophoresis of on-chip LAMP products using a series of diluted DNA template solutions. Lanes 1-9: 100 bp marker, 5×10⁵, 5×10⁴, 5×10³, . . . , 5×10⁰, 5×10⁻¹ DNA copies of the template per LAMP zone, NC.

FIGS. 7a-c. Investigation of on chip bacterial lysis performance. (a) Normalized lysis performance at different ratio of artificial sample to bacterial lysis buffer. (b) Gel electrophoresis test of the released DNA after the bacterial lysis; (c) Normalized on-chip bacterial lysis performance based on the nucleic acid concentration after the bacterial lysis. Method 1 (M1), introducing 6 μL the artificial sample/bacterial lysis buffer mix (1:1) together on chip to lysis for 10 min; Method 2 (M2), introducing 6 μL artificial sample/saline mix (1:1) on chip, then introducing 6 μL bacterial lysis buffer to lysis for 10 min; Method 3, introducing 6 μL artificial sample/saline mix (1:1) on chip, heating at 95° C. to lysis for 10 min.

FIGS. 8a-e. On-chip LAMP reaction and detection of B. pertussis pathogenic microorganism using an artificial sample by a portable UV light pen (a) and fluorescence microscopy (b). Strong fluorescence was observed in B. pertussis and PC LAMP zones, but not in NC zones. (c) Gray value of the LAMP products measured by ImageJ; (d) Fluorescent intensity of the LAMP products measured by fluorescence microscope. (e) Gel electrophoresis of on-chip LAMP products. Lanes 1-4: 100 bp ladder, B. pertussis LAMP products, PC, NC. Ladder-pattern DNA bands were observed in B. pertussis LAMP products and PC, whereas no DNA bands were observed in NC. A tiny amount of B. pertussis bacterial colonies were added in the nasopharyngeal swabs to form an artificial sample.

FIG. 9. The schematic of the cellphone-based detection system. After on-chip LAMP reactions, a portable UV light pen was applied to shine LAMP products on the biochips to obtain the visual detection results. The fluorescent images were captured by a cellular phone camera and were further processed with an NIH software ImageJ to obtain the gray values of each LAMP zone.

FIGS. 10a-c. Direct instrument-free detection of clinical samples #2, #8 and #10. (a) Fluorescence images of LAMP products from samples #2, #8 and #10, as well as the NC. (b) Gray values measured by ImageJ; (c) Gel electrophoresis analysis.

### DESCRIPTION

Loop-mediated isothermal amplification (LAMP) is a nucleic acid amplification technique that can amplify target DNA at a constant temperature between 60° C. and 65° C. within one hour (Tomita et al., Nature protocols 2008, 3, 877-882). Kamachi et al. developed the LAMP method for pertussis diagnosis and evaluated its high sensitivity and specificity (Kamachi et al., Journal of clinical microbiology 2006, 44, 1899-1902). However, this LAMP diagnosis method still requires specific instruments such as thermocyclers and centrifuges in a well-equipped laboratory and complicated sample preparation procedures. Those limitations hinder its broad applications for pertussis diagnosis especially in low-resource settings, and cause POC testing challenging to achieve.

The inventors have developed a POC microfluidic biochip (“biochip”) for rapid, highly sensitive and specific pertussis diagnosis. The POC biochip can be used in various venues such as physician's office, schools, hospitals, and low-resource settings so that rapid prevention and treatment of pertussis can be achieved. The POC biochip based pertussis diagnosis is low-cost, rapid, highly sensitive and specific.

In certain embodiments the POC biochip based pertussis diagnosis is instrument-free, and does not require any external equipment or instruments such as thermocyclers or centrifuges, or even AC electricity. The inventors have developed a fully battery-powered portable heating device that is compatible with the biochip for LAMP reaction. The results of an assay performed using the biochip can be detectable under a portable UV light pen by the naked eye, or recorded by a smartphone camera.

In certain embodiments a lysis buffer can be used when performing the assay on clinical samples so that the clinical samples can be directly tested without complicated or time-consuming sample preparation and DNA isolation procedures. One-hundred clinical samples were tested by using the POC biochip. The test results were verified by comparison with the qPCR test results.

Certain embodiments are directed to a fully battery-powered low-cost spectrophotometric system for quantitative POC analysis on a microfluidic chip. The spectrophotometric system is fully battery-powered does not require external AC electricity. This feature is significant for POC analysis and in-field detection in resource-poor settings. The spectrophotometric system described herein exhibits high detection sensitivity. LAMP detection and subsequent nucleic acid analysis will enable wide applications of the spectrophotometric system due to the widely-used DNA testing techniques.

In certain embodiments a battery-powered spectrophotometric system can include one or more of (i) an optional light source, (ii) a microfluidic device, (iii) a heater, (iv) an optional detector, and (v) a battery as a power source.

One or more device holder can be included that is compatible with one or more microfluidic device. The device holder can secure the microfluidic device and provide for proper alignment for processing and detection purposes. The device holder can form a microfluidic device port that accept a microfluidic device and position the device inside the holder during microfluidic chip processing. In one aspect the microfluidic device port aligns the microfluidic device with a heating element. In certain aspects the device holder can comprise a user interface and a power switch.

In certain embodiments of a microfluidic device can be configured for single- or multiplexed pathogen detection. One such device can have three or more layers. The top layer can be a polymer layer used for reagent delivery. Microchannels (e.g., length 9.5 mm, width 100 μm, depth 100 μm) can be formed in the top layer that connect various zones or wells of the device. Also formed in the top layer can be an inlet reservoir (e.g., diameter 1.0 mm, depth 1.5 mm). The middle layer can be a polymer layer having two or more detection zones, outlet reservoirs (e.g., diameter 1.0 mm, depth 1.5 mm) and microchannels (e.g., length 9.5 mm, width 100 μm, depth 100 μm). In certain aspects the detection zone can be a LAMP zone(s) that can be used for LAMP reaction and detection. The bottom layer can be a support layer (e.g., a glass slide or other support (length 75 mm, width 25 μm, depth 1.0 μm). Different detection zones can be used for negative control (NC), positive control (PC), and pathogen detection.

The detection portion or amplification zone of a microfluidic device can comprise specific primers and/or specific probes for target pathogens. A positive control DNA can be pre-loaded or supplied during the processing of a sample in a detection zone. In certain aspects a detection zone can be loaded with 1, 2, 3, 4, or more primer pairs. In certain aspects a different set of primers can be in various separate amplification or detection zones. In certain aspects amplification and detection are performed in the detection/amplification zone. In a further aspect, an amplification reaction product can be transferred to a separate detection zone via microchannel. A microfluidic device can be configured to transport a reaction mixture and/or sample from an inlet to fill the detection zone(s). In certain aspects a filter is included in the device and positioned such that a sample being applied to the device is filtered prior to being transported to a detection zone. After filling, the inlet and outlets can be sealed, e.g., with epoxy. The microfluidic device can be inserted into the holder through the microfluidic device port where it can be heated using a heater. Amplification can then then performed at an appropriate temperature an appropriate amount of time. Microfluidic devices and systems can be configured to perform a number of different analytical and/or synthetic operations within the confines of very small channels and chambers that are disposed within a microfluidic device. Multiplexing by providing for more than one sample or more than one amplification reaction can substantially increase throughput, so that the operations of the system are carried out in parallel.

Microfluidic devices and systems are well suited for parallelization or multiplexing because large numbers of parallel analytical fluidic elements can be combined within a single integrated device that occupies a relatively small area. A multiplexing device will comprise a plurality of channels and microwells that are configured to analyze a number of different analytes, such as pathogens.

A microfluidic device can comprise a nucleic acid amplification zone or detection zone(s), microchannels, and ports. Each detection zone can have one or more detectable probes or detection compounds.

In certain aspects the amplification zone or detection zone can be sealed, for example with a tape layer, a cap, or mineral oil to prevent liquid evaporation. DNA in a sample(s) can be isothermally amplified by LAMP or a similar process (Ahmad et al. (2011) Biomed Microdevices, 13(5): 929-37). In certain aspects, a portable heating unit can be included in the microfluidic device holder. In one aspect the heating unit can include a proportional-integral-derivative (PID) temperature controller (Auber Inst, GA.), a thermocouple (Auber Inst.), and a heating film (Omega, Conn.). During processing a target analyte (e.g., a target nucleic acid) can be labeled with fluorophores or associated with a labeled probe for fluorescence detection. In other aspects the detection reagent can be activated by the amplification process, for example calcein. Calcein (also known as fluorexon, fluorescein complex) is a fluorescent dye with excitation and emission wavelengths of 495/515 nm, respectively. Calcein self-quenches at concentrations above 70 mM. It is used as a complexometric indicator for titration of calcium ions with EDTA, and for fluorometric determination of calcium.

In certain embodiments the microfluidic device is configured for nucleic acid amplification using LAMP or other isothermal nucleic acid amplification methods. In certain aspect a LAMP method can use Bacillus stearothermophilus DNA polymerase, a thermally-stable enzyme with high displacement ability over the template-primer complex (Saleh et al. (2008) Dis Aquat Organ, 81(2): 143-51; Notomi et al. (2000) Nucleic Acids Res, 28(12): E63). The LAMP amplification technique allows nucleic acid amplification to be carried out under thermally constant conditions, eliminating the use of expensive and cumbersome thermal cycler equipment in low-resource settings.

Certain embodiments incorporate a miniaturized portable fluorescence detection system using a light emitting diode (LED), such as violet LED (Tsai et al. (2003) Electrophoresis, 24(17): 3083-88), a UV LED, or a laser pointer. The wavelength of 532 nm from a green laser pointer is a good fit with the excitation wavelength of one of the common probes—Cy3, but other combinations of light source and fluorophore can be used.

Certain embodiments incorporate a visual fluorescent or a colorimetric detection method. Mori et al (2001) observed that during the LAMP amplification process, a magnesium pyrophosphate precipitate was formed as a turbid by-product of the nucleic acid amplification process (Mori et al. (2001) Biochem Biophys Res Commun, 289(1): 150-54). This precipitate forms only when the targeted DNA is present in the LAMP amplification process, such that the presence of the pyrophosphate can serve as an indicator of the presence of a target. In certain aspects an intercalating dye can be used to detect product amplification (Ji et al. (2010) Poult Sci, 89(3): 477-83). In certain embodiments a filtration layer can be included to remove red blood cells in order to avoid detection inference in subsequent steps.

Certain configurations of the system can include configurations for receiving microfluidic devices having multiple detection zones. In certain aspects different detection zones will have different primers or different control DNA. When an appropriate target is present a detectable signal is generated, detected, and processed by the system or observed by a user. In certain aspect the system will comprise a computer or controller to receive detection data, process the detection data, generate a result, and present the result to receiver. The receiver can be a human or other electronic device configured to manage such information.

In certain embodiments a microfluidic device is configured for B. pertussis detection/diagnosis in a laboratory or home setting. In other embodiments the system is configured to provide a POC device for field diagnosis.

Probes can be coupled to a variety of reporter moieties. Reporter moieties include fluorescent reporter moieties that can be used to detect probe binding to or amplification of a target. Fluorophores can be fluorescein isothiocyanate (FITC), allophycocyanin (APC), R-phycoerythrin (PE), peridinin chlorophyll protein (PerCP), Texas Red, Cy2, Cy3, Cy3.5, Cy5, Cy5.5, Cy7; or fluorescence resonance energy tandem fluorophores such as PerCPCy5.5, PE-Cy5, PE-Cy5.5, PE-Cy7, PE-Texas Red, and APC-Cy7. Other fluorophores include, Alexa Fluor® 350, Alexa Fluor® 488, Alexa 25 Fluor® 532, Alexa Fluor® 546, Alexa Fluor® 568, Alexa Fluor® 594, Alexa Fluor® 647; BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665; Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, and tetramethylrhodamine, all of which are also useful for fluorescently labeling nucleic acids or other probes or targets.

In certain aspects the fluorescence of a probe can be quenched. Quenching refers to any process that decreases the fluorescence intensity of a given substance. A variety of processes can result in quenching, such as excited state reactions, energy transfer, complex-formation, and collisional quenching. The chloride ion is a well-known quencher for quinine fluorescence. Typically quenching poses a problem for non-instant spectroscopic methods, such as laser-induced fluorescence, but can also be used in producing biosensors. In certain aspects the fluorescence of a labeled probe that is not bound to its target is quenched, wherein upon binding to its target the fluorescence is recovered and can be detected. The labeled probe is complexed with a quenching moiety in the detection zone. Once the probe binds its target the fluorescence is recovered. Target binding results in increased fluorescence.

In certain embodiments, the invention concerns portable, rapid and accurate POC systems for detecting microbes, including B. pertussis. The term “microorganism” or “microbe” as used in this disclosure includes bacterium. In certain aspects a pathogenic or potentially pathogenic microbe can be detected. In certain aspects the system can be configured to detect a variety of bacteria simultaneously. A bacterium can be an intracellular, a gram positive, or a gram negative bacteria. In a further aspect, bacteria include, but is not limited to a B. pertussis, B. parapertussis, or B. holmesii bacteria.

### I. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Chemicals and Materials

The LAMP primers (Integrated DNA Technologies, Coralville, Iowa) targeting the PT promoter region of B. pertussis are shown in Table 1 (Kamachi et al., Journal of clinical microbiology 2006, 44, 1899-1902). Loopamp DNA amplification kit and Loopamp fluorescence detection reagent (calcein) were purchased from Eiken Co. Ltd., Japan. The Loopamp reaction mixture contained 20 mM Tris-HCl (pH 8.8), 10 mM KCl, 8 mM MgSO₄, 10 mM (NH₄)₂SO₄, 0.1% Tween 20, 0.8 M Betaine, 0.5 mM MnCl₂, 1.4 mM dNTPs, 8U Bst Polymerase, 1.6 μM each of the inner primer (FIP/BIP), 0.2 μM each of the outer primer (F3/B3), 0.4 μM each of the loop primer (LF/LB). DNA isolation kit and LAMP product purification kit were purchased from Qiagen (Valencia, Calif.). Bacterial lysis buffer contained 4 M urea, 0.1% triton and 50 mM Tris buffer (pH 7.5). Clinical samples are from the Department of Pathology and Laboratory Medicine at Children's Hospital Los Angeles.

Polydimethylsiloxane (PDMS, Sylgard 184) and the curing agent were obtained from Dow Corning (Midland, Mich.); Whatman #1 chromatography paper and Epoxy glue were purchased from Sigma (St. Louis, Mo.) and ITW Devcon (Danvers, Mass.), respectively.

All other chemicals were purchased from Sigma (St. Louis, Mo.) and used without further purification, unless stated otherwise. Unless otherwise noted, all solutions were prepared with ultrapure Milli-Q water (18.2 Macm) from a Millipore Milli-Q system (Bedford, Mass.).

TABLE 1
LAMP primers for B. pertussis LAMP assay
(Kamachi et al., Journal of clinical
microbiology 2006, 44, 1899-1902)

| Primer | Sequences (5′-3′) | bp |
|---|---|---|
| FTP | TTGGATTGCAGTAGCGGGATGTGCATGCGTGCAGATTCG | 41 |
| | TC (SEQ ID NO: 1) | |
| BIP | CGCAAAGTCGCGCGATGGTAACGGATCACACCATGGCA | 38 |
| | (SEQ ID NO: 2) | |
| F3 | CCGCATACGTGTTGGCA (SEQ ID NO: 3) | 17 |
| B3 | TGCGTTTTGATGGTGCCT (SEQ ID NO: 4 | 18 |
| FL | ACGGAAGAATCGAGGGTTTTGTAC (SEQ ID NO: 5) | 24 |
| BL | GTCACCGTCCGGACCGTG (SEQ ID NO: 6) | 18 |

Microorganism culture and DNA preparation. B. pertussis (ATCC 9797) was obtained from American Type Culture Collection (ATCC, Rockville, Md.), which was grown on Regan-Lowe Charcoal Agar plates (BD, Sparks, Md.) supplemented with casamino acid. The microorganisms were incubated at 37° C. for 5 min. DNA was extracted by using Qiagen DNA Mini kit following a protocol from the manufacturer.

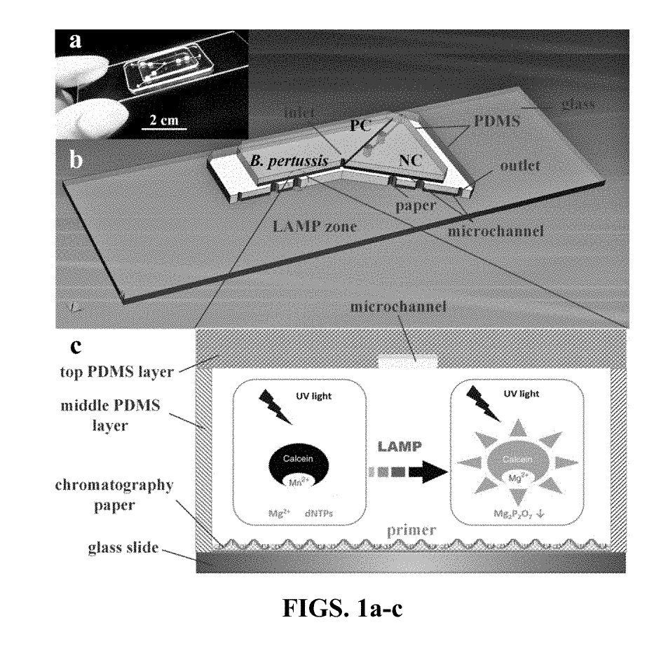

Microfluidic biochip design and fabrication. An embodiment of a microfluidic device is shown in FIG. 1, the microfluidic biochip can comprise three layers, two PDMS layers on the top of a glass slide. The top PDMS layer can contain an inlet reservoir (diameter 1.0 mm, depth 1.5 mm) and microchannels (width 100 μm, depth 100 μm) is used for reagent delivery. The middle PDMS layer can contain 3 outlet reservoirs (diameter 1.0 mm, depth 1.5 mm), microchannels, and 6 LAMP zones (diameter 2.0 mm, depth 1.5 mm) that can be used for LAMP reaction and detection. Different LAMP zones are used for negative control (NC, omission of LAMP primers), positive control (PC) and B. pertussis detection, respectively. PC template DNA and its primer mix (PM) were provided by the Loopamp DNA amplification kit. In addition, a piece of Whatman #1 chromatography paper with a diameter 2.0 mm cut by using a laser cutter (Epilog Zing 16, Golden, Colo.) is placed inside each LAMP zone. The bottom glass layer is used for structure support.

PDMS films were prepared by following the standard soft lithography procedures (Xia and Whitesides, Rev. Mater. Sci. 1998, 1998, 31). Briefly, the liquid PDMS base and the curing agent were mixed at a weight ratio of 10:1. Then the PDMS precursor mixture was poured into a petri dish, degassed in a vacuum desiccator for ˜30 min, and incubated at 85° C. for 3 h. Unlike the commonly used PDMS moulding, microchannels were directly created on top of the PDMS film via ablation using the laser cutter. Inlet reservoir in the top PDMS layer, outlet reservoirs and LAMP zones in the middle PDMS layer were excised using biopsy punches. After 30 seconds exposure in an oxidizing air Plasma Cleaner (Ithaca, N.Y.), PDMS films and the glass slide were face-to-face sandwiched to bond irreversibly. After the biochip assembly, the specific LAMP primers for B. pertussis and PC DNA sequences were pre-loaded into the LAMP zones with paper inside. Thus, the microfluidic biochip became ready for use.

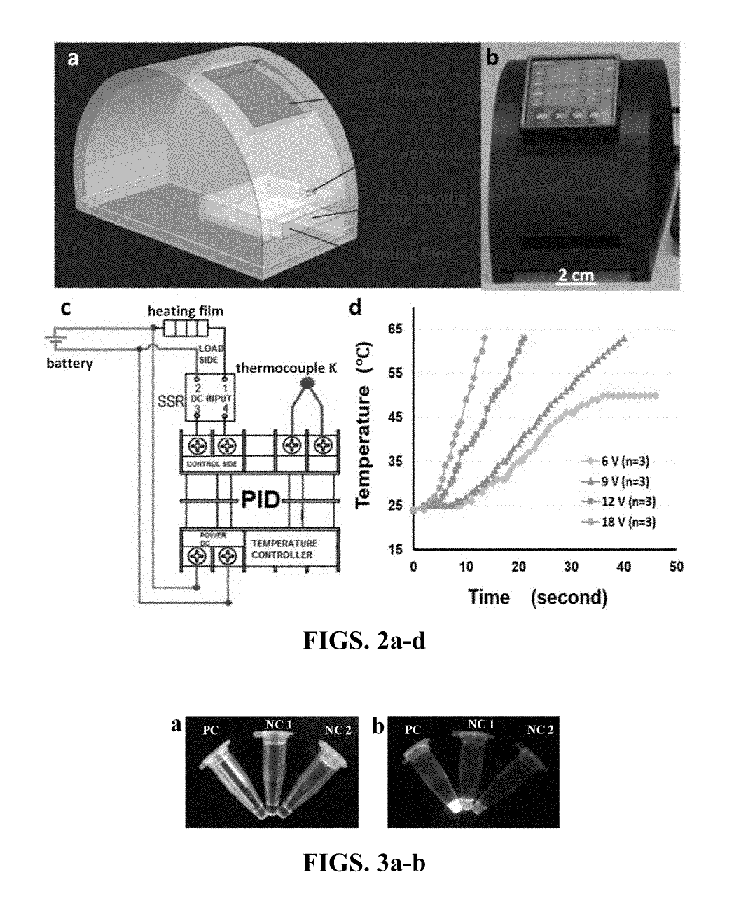

Portable battery-powered heater for on-chip LAMP reaction. After the LAMP mix was prepared in a biosafety cabinet, the 26 μL LAMP reaction mix was introduced to the biochip from the inlet reservoir to fill the LAMP zones. After the inlet and outlets reservoirs were sealed with Epoxy, the biochip was heated by using a portable battery-powered heater (for an example see FIG. 2) at 63° C. for 45 min for LAMP reactions, followed by the termination of LAMP reactions at 95° C. for 2 min.

FIG. 2a is a 3D schematic of the holder for the portable battery-powered heater, illustrating the location of different components inside. The microfluidic chip rests above the heating film. FIG. 2b is a photograph of the portable-battery powered heater. FIG. 2c shows the schematic of the portable-battery powered heater with a PID-based temperature controller. The PID controller is used to control the heater under precise temperature ranges. A thermocouple K is included in this system in order to monitor the temperature in real time while operating, providing feedback to the PID controller. The output signal is provided by a solid state relay to increase or decrease the temperature of the heater. To meet the required detection speed without sacrificing battery life or exceeding in weight, cyclic battery testing was performed at four different voltages 6, 9, 12 and 18 volts. At 6 volts the goal temperature could not be reached. At 9, 12, and 18 volts the goal temperature of 63° C. was reached within 40, 22, and 13 seconds respectively (FIG. 2d). Therefore, a 9-volts battery was determined to be used as the power supply since it would meet the required detection speed of two minutes without adding excessive time to reach the optimal detection temperature of 63° C. for the LAMP reaction. This approach adds only 45.6 g to the design; thus maintaining a low weight required for POC detection. Battery life for the 9-volts battery was also estimated to be 2.3 hours, using the following formula: Battery Life=Watt Hours/(Voltage×Current in Ampere), total meeting the required design input. The total material cost of this heater was about $60.

Confirmation tests. After LAMP reactions, the generated fluorescence images were captured by using a cellular phone camera (iPhone 5), and the captured images were processed by using the NIH software ImageJ to obtain grey values for analysis. Results were further confirmed by a high-sensitivity Nikon Ti-E fluorescence microscope (Melville, N.Y.) that was equipped with a motorized stage and a cooled CCD camera to measure the fluorescence intensities, using appropriate FITC optical filters (Ex=495 nm; Em=520 nm). LAMP products were collected from the outlet of the biochip for further confirmatory tests using gel electrophoresis (Sub-Cell GT, Bio-Rad, CA) by applying 90 volts for 1 hour in 1.5% agarose gel.

B. Results and Discussions

Off-chip LAMP reaction (SI). Negative controls of omission of DNA template (NC1) and omission of LAMP primers (NC2) were tested off-chip. As showed in FIG. 3, after LAMP reaction, the PC was a green color under daylight and bright green fluorescence under a portable UV light. On the contrary, both NCs showed the same results as before LAMP reaction, without notable color change or fluorescence. Herein omission of LAMP primers was adopted as the main NC for the subsequent on-chip LAMP reaction experiments.

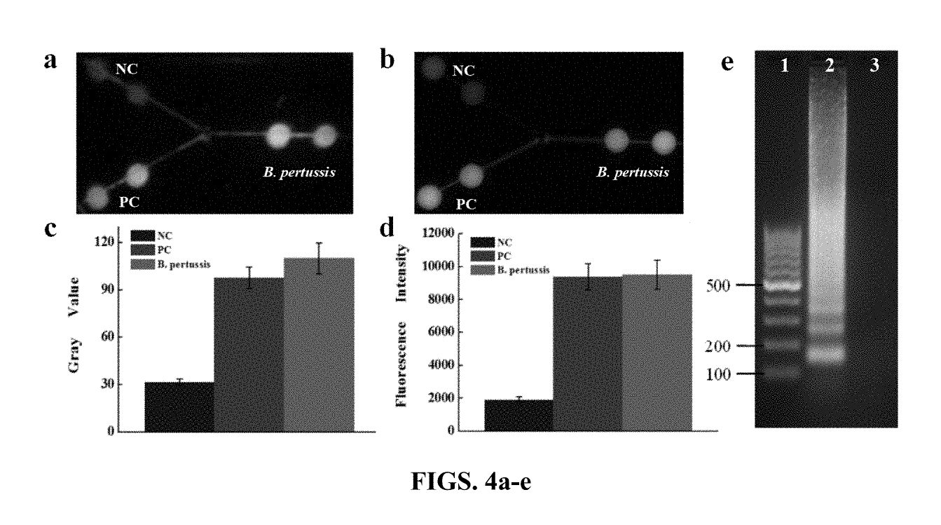

On-chip LAMP reaction. The feasibility of the PDMS/paper hybrid microfluidic chip for B. pertussis detection was tested by using extracted DNA templates. The detection principle is illustrated in FIG. 1c. Before LAMP reaction, fluorescence of the reagent calcein in the reaction mix is quenched by manganese ions. During LAMP reaction, the by-product pyrophosphate ion forms a complex with a manganese ion. As a result, calcein is free to combine magnesium ions, producing bright fluorescence. After LAMP reaction the results can be observed by the naked eye based on the fluorescence of the LAMP zones under a portable UV light pen. As shown in the fluorescence image captured by a cellphone camera (FIG. 4a), under a portable UV light pen, B. pertussis sample and PC showed bright green fluorescence while NC only showed weak background. Then the image was processed by software ImageJ to obtain the gray value which indicates of the brightness of a pixel. As shown in FIG. 4c, the gray value difference of more than 3 folds between the NC and PC/B. pertussis was observed.

The result was confirmed by high-sensitivity fluorescence microscopy. Similarly, strong fluorescence was observed in B. pertussis and PC LAMP zones, but not in NC zones (see FIG. 4b). The fluorescence intensity of the B. pertussis LAMP products was about 4.5 times higher than that of the NC (see FIG. 4d). Subsequently, the results were confirmed by gel electrophoresis using the extracted LAMP products from different outlets, as shown in FIG. 4e. The multiple ladder-pattern DNA bands (lane 2) confirmed the success of the on-chip LAMP reaction.

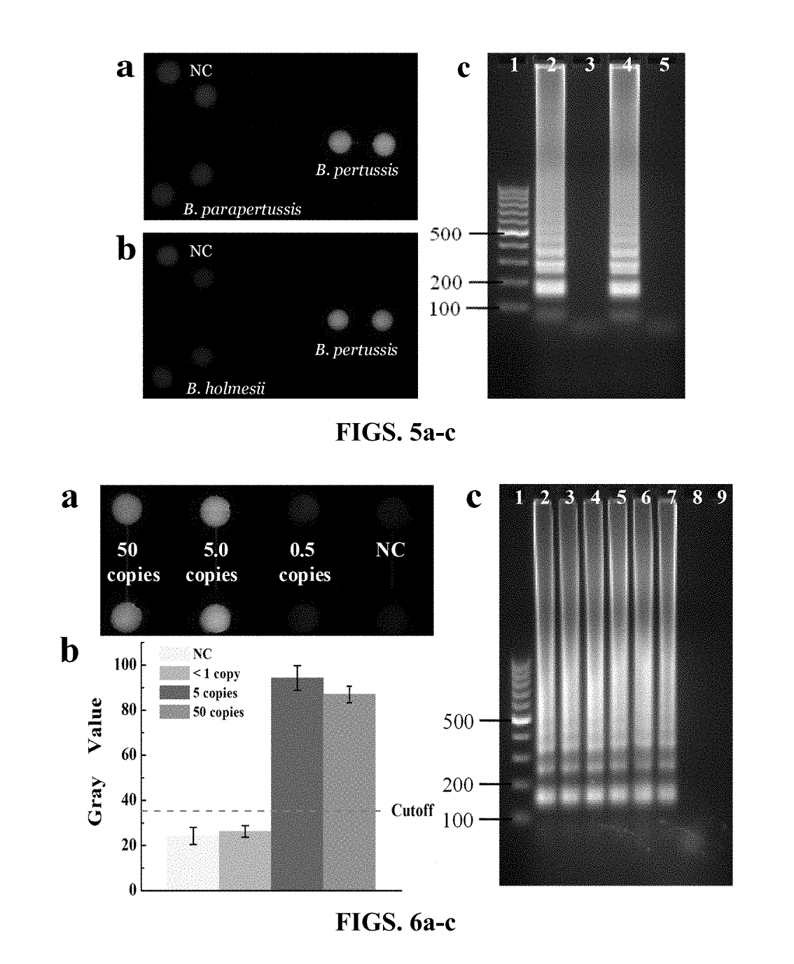

Specificity detection. The identification of the exact B. pertussis bacteria is important to apply the accurate specific treatment and antibiotic for pertussis. B. parapertussis and B. holmesii are similar species associated with respiratory infections in humans, which may also cause a pertussis-like syndrome. Therefore, the specificity of the approach for B. pertussis detection with its similar species B. parapertussis and B. holmesii was investigated. Except the NC LAMP zones, all the other LAMP zones were preloaded with B. pertussis primers. After that, different DNA samples of B. pertussis, and B. parapertussis and B. holmesii were introduced into different LAMP zones separately. The results are shown in FIGS. 5a and 5b. It demonstrated that only the LAMP zones with B. pertussis DNA sample and its corresponding LAMP primers exhibited strong fluorescence signal. In contrast, for the LAMP zones with B. parapertussis and B. holmesii DNA samples was observed similarly to the NC. The results indicated the high specificity of the approach for the detection of B. pertussis. Gel electrophoresis analysis further confirmed the results. As shown in FIG. 5c, only the LAMP products extracted from the LAMP zones with B. pertussis DNA sample exhibited DNA sizing ladder.

Limit of detection (LOD). By using a serial of 10-fold diluted B. pertussis DNA samples, the LOD was tested. The initial copy number of the DNA template was ranged from 5×10⁵, 5×10⁴, 5×10³, . . . , 5×10⁰, 5×10⁻¹ copies of the DNA template per LAMP zone. As shown in FIG. 6a, strong fluorescence of the LAMP products was observed even when the initial DNA template was as low as 5 copies per LAMP zone. However, when the initial DNA template was less than 1 copy, the fluorescence of the LAMP products was as dim as the NC. The cutoff gray value was calculated to be 35.5 on the basis of 3-fold standard deviations of the mean gray value of the NC, as shown in FIG. 6b. The gray value of the LAMP products from 5 copies of initial DNA template was much higher than that of the cutoff. But the LAMP products from less than 1 copy of initial DNA template was below the cutoff gray value. The result was further confirmed by gel electrophoresis (see FIG. 6c). Therefore, it was concluded that the LOD of the microfluidic approach for B. pertussis was as low as ˜5 copies per LAMP zone.

On-Chip lysis performance. The tests described above were conducted by using isolated DNA samples. To avoid complicated and time-consuming DNA isolation procedures, a simple centrifuge-free approach was developed for direct detection of microorganisms by integrating an on-chip bacteria lysis procedure for the following LAMP reaction and detection. Instead of using extracted DNA template, an artificial sample was prepared by adding B. pertussis bacteria in nasopharyngeal swabs to mimic the real clinic samples to investigate and optimize the bacterial lysis performance.

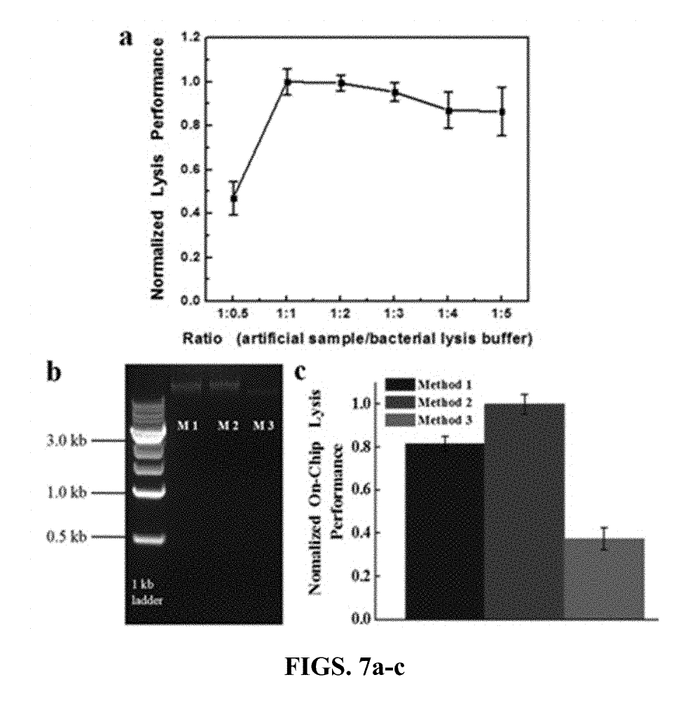

The ratio between the artificial samples and bacterial lysis buffer were optimized with a ratio ranging from 1:0.5 to 1:5. Different ratio of a mix of artificial samples with the bacterial lysis buffer was placed at room temperature for 10 min to lysis the pathogenic microorganisms to release DNA. After that, the nucleic acid concentration was assessed with Nanodrop and calculated the DNA amount for each solution, on the basis of which their normalized lysis performance was shown in FIG. 7a. It demonstrated that higher lysis performance could be achieved when the ratio between the artificial samples and the bacterial lysis buffer was below 1.0. However, a smaller ratio meant more bacterial lysis buffer added, which could decrease the concentration of the artificial samples. Therefore, the ratio of 1.0 between the artificial samples and the bacterial lysis buffer was used in the following tests.

The lysis efficiency was compared by using different bacteria lysis methods with same amount of artificial samples. Method 1 and method 2 are adding the artificial samples with or followed by on-chip bacteria lysis buffer at room temperature for 10 min. Method 3 is heating the on-chip artificial samples at 95° C. for 10 min. After the bacterial lysis process, gel electrophoresis and Nanodrop were used to investigate their lysis performance, as shown in FIGS. 7b-c. The bacteria lysis buffer based lysis methods (Method 1 and 2) showed brighter DNA bands in the gel image and higher normalized lysis performance on the basis of DNA concentration after lysis, which indicated that the simple bacteria lysis buffer based lysis methods could effectively lysis the pathogenic microorganism to release DNA. Therefore, Method 1 and Method 2 by using bacterial lysis buffer are used for the following LAMP reaction and detection.

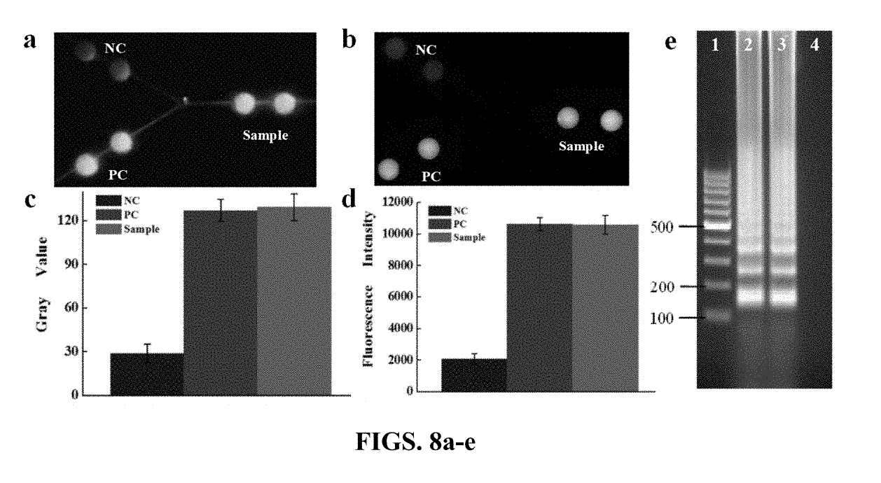

Instrument-free direct detection of pathogenic microorganisms. LAMP reaction and detection for B. pertussis was carried out using the optimized on-chip bacterial lysis buffer based lysis Method 2. Isolated DNA samples were introduced from the traditional sample preparation in the PC LAMP zones and the artificial samples with on-chip bacterial lysis buffer based lysis Method 2 in the sample LAMP zones, respectively. The results showed that strong fluorescence could be produced by using the artificial samples as by using isolated DNA samples (PC), with the confirmation of gel electrophoresis (see FIG. 8). This is very significant because it indicated that the microfluidic approach described herein has the potential for direct detection of clinic samples without using any equipment such as centrifuge, for complicated and time-consuming DNA isolation pretreatment.

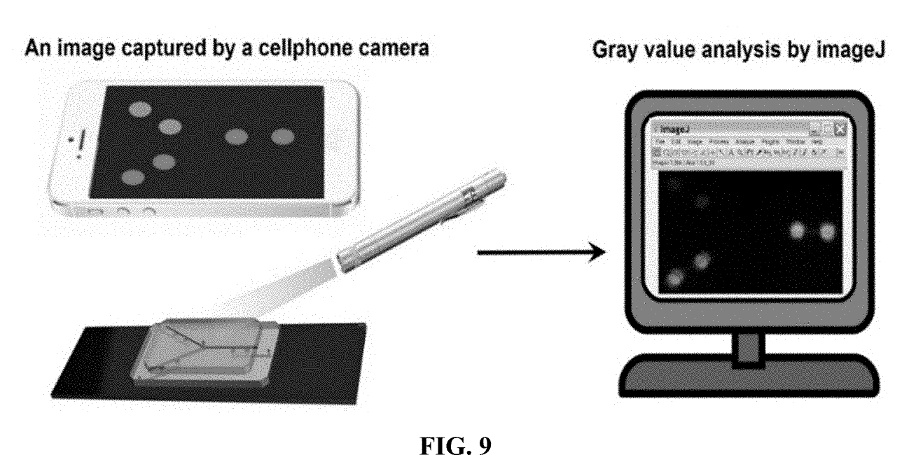

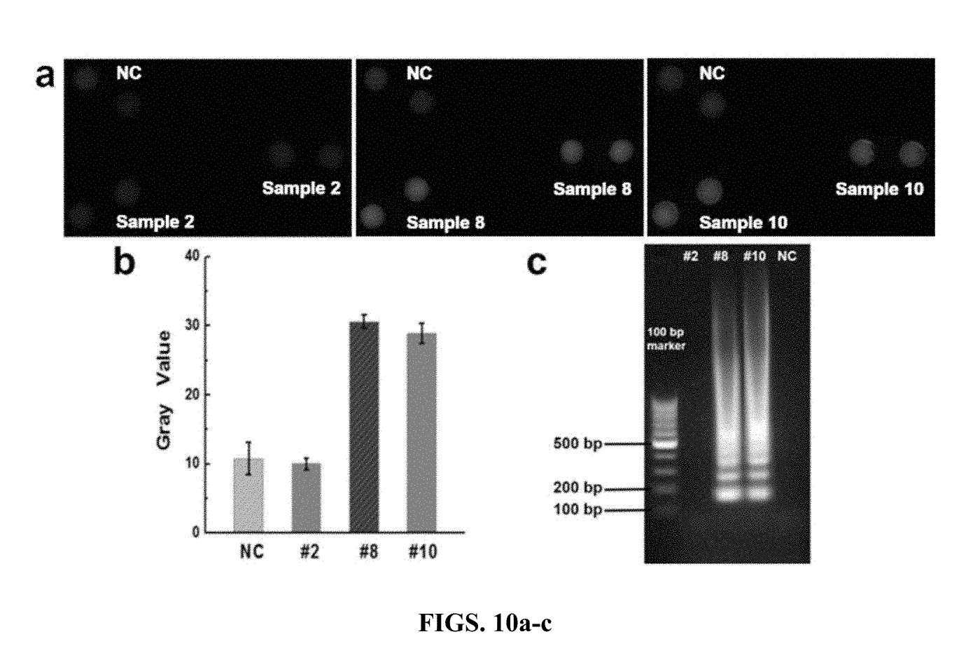

Validation using clinical samples. One hundred de-identified (no information about gender, age or ethnicity) clinical samples from pediatric patients were obtained and tested. The schematic of the cellphone-based detection system is demonstrated in FIG. 9. The generated fluorescence of LAMP zones after on-chip LAMP reactions was captured by a cellular phone camera (e.g., iPhone 5) under a portable UV light pen. The cellphone camera captured images were processed with the NIH software ImageJ to obtain the gray values of each LAMP zone to indicate the brightness of the measured areas. The performance of the hybrid microfluidic biochip for rapid clinical sample diagnosis was first validated by using randomly selected sample #2, #8, and #10. According to the real-time PCR test that is used as a reference assay, sample #2 is negative, and samples #8 and #10 are positive. The captured fluorescence images in FIG. 10a shows that samples #8 and #10 exhibited bright green fluorescence while sample #2 and NC only showed very weak fluorescence. As shown in FIG. 10b, a ˜3 folds difference between the samples #8/#10 and the sample #2/NC was observed. Subsequently, the LAMP products were collected and applied for gel electrophoresis analysis to confirm the results. FIG. 10c exhibits that there was no DNA band from the negative clinical sample #2. On the contrary, the multiple ladder-pattern DNA bands from samples #8 and #10 confirmed the success of the on-chip LAMP reaction. All those observations indicated that sample #8 and #10 were positive and sample #2 was negative. The results were consistent with the real-time PCR test. The remaining clinical samples were tested and compared with the real-time PCR test results. All the 53 positive samples according to the real-time PCR were tested to be positive as well by the on-chip LAMP method. For the 47 negative samples according to the real-time PCR test, it was found that 45 of them were negatives and 2 of them were positives based on the on-chip LAMP method. The sensitivity and specificity were calculated to be 100% and 96%, respectively.

1. A battery-powered loop-mediated isothermal amplification (LAMP) system comprising:
a microfluidic device comprising at least one amplification zone containing amplification primers that differentially amplify Bordetella pertussis (B. pertussis) DNA;
a battery operated heater configured to maintain the amplification zone at an amplification temperature; and
a simple detection system.

2. The system of claim 1, wherein qualitative results for B. pertussis detection can be visible to the naked eye under a portable UV light source or recorded by a smartphone camera, without the use of specialized instruments.

3. The system of claim 1, wherein detection results can be quantified by a low-cost portable battery-powered spectrometer.

4. The system of claim 1, wherein the microfluidic device comprises a positive control and a negative control for B. pertussis DNA amplification.

5. The system of claim 1, wherein the microfluidic device is composed of paper and polymers as the device substrate.

6. The system of claim 5, wherein paper is used to preload primers, enabling longer shelf life.

7. The system of claim 1, wherein the microfluidic device is configured to receive two or more samples.

8. The system of claim 1, further comprising a user interface.

9. The system of claim 1, wherein the battery is a 9-volt battery.

10. A method of detecting Bordetella pertussis in a patient sample comprising:
performing bacterial lysis on a sample suspected of having B. pertussis bacteria;
introducing the lysed sample suspected of having B. pertussis bacteria into a microfluidic device configured to specifically amplify B. pertussis DNA;
incubating the microfluidic device at a temperature for LAMP amplification of B. pertussis DNA; and
detecting the presence or absence of B. pertussis DNA based on DNA amplification.

11. The method of claim 10, wherein the LAMP amplification temperature is 55° C. to 65° C.

12. The method of claim 10, wherein the microfluidic device is configured with a positive control and a negative control for B. pertussis DNA amplification.

13. The method of claim 10, further comprising exposing the sample to a bacterial lysis solution, without centrifuges or other equipment for cell lysis.

14. The method of claim 10, wherein the sensitivity and specificity for clinical sample testing are 100% and 96%, respectively.
